(19) United States
(12) Patent Application Publication
(10) Pub. No.: US 2008/0004222 A1
(43) Pub. Date: Jan. 3, 2008

(54) Antitumorigenic Drug Combination

(76) Inventor: Jack Henkin, Highland Park, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/770,977

(22) Filed: Jun. 29, 2007

Related U.S. Application Data
(60) Provisional application No. 60/806,161, filed on Jun. 29, 2006.

(51) Int. Cl. A61K 38/17; A61K 38/08

(52) U.S. Cl. 514/12; 514/16

(57) ABSTRACT

Compositions comprising drugs having additive antitumorigenesis activity and methods of treatment using the combinations is disclosed.

This application claims priority to the provisional application Ser. No. 60/806,161 filed on Jun. 29, 2006.

### FIELD OF THE INVENTION

The invention relates to compositions comprising drugs having additive antitumorigenesis activity and methods of treatment using the combinations.

### BACKGROUND OF THE INVENTION

The hallmark of neuroblastoma (NB) is heterogeneity, with the likelihood of tumor progression varying widely according to age at diagnosis and extent of disease. In addition to these clinical factors, biologic markers such as MYCN gene status, tumor cell ploidy, and tumor histology have also been shown to be strongly predictive of risk of relapse. Modern treatment strategies are stratified according to these clinical and biological classifiers, and substantial progress has been made in the treatment of children with low- and intermediate-risk NB with reduced therapy approaches. However, more effective therapy is still needed for children with high-risk disease.

### BRIEF DESCRIPTION OF THE FIGURE

FIG. 1 shows comparative antitumorigenesis of VPA and N—-Ac-Sar-Gly-Val-D-alloIle-Thr-Nva-Ile-Arg-Pro-NHCH₂CH₃ compared to VPA or N—Ac-Sar-Gly-Val-D-alloIle-Thr-Nva-Ile-Arg-Pro-NHCH₂CH₃ in NB cell limes and xenografts.

### SUMMARY OF THE INVENTION

Accordingly, one embodiment of this invention pertains to compositions for treating cancer in a mammal with measurably additive antitumorigenesis, said compositions comprising therapeutically effective amounts of a histone deacetylase (HDAC) inhibitor and a peptidomimetic of the second of the three Type-1 repeats of thrombospondin-1 (TSP-1) or a salt, prodrug or salt of a prodrug of either or both.

Still another embodiment pertains to separate compositions to be administered together for treating cancer in a mammal with measurably additive antitumorigenesis, one comprising a therapeutically effective amount of a HDAC inhibitor or a salt, prodrug, or salt of a prodrug thereof, and the other comprising a peptidomimetic of the second of the three Type-1 repeats of TSP-1 or a salt, prodrug or salt of a prodrug thereof.

Still another embodiment pertains to compositions for treating cancer in a mammal with measurably additive antitumorigenesis, said compositions comprising therapeutically effective amounts of a HDAC inhibitor and N—Ac-Sar-Gly-Val-D-alloIle-Thr-Nva-Ile-Arg-Pro-NHCH₂CH₃ or a salt, prodrug or salt of a prodrug of either or both.

Still another embodiment pertains to separate compositions to be administered together for treating cancer in a mammal with measurably additive antitumorigenesis, one comprising a therapeutically effective amount of a HDAC inhibitor or a salt, prodrug or salt of a prodrug thereof, and the other comprising N—Ac-Sar-Gly-Val-D-alloIle-Thr-Nva-Ile-Arg-Pro-NHCH₂CH₃ or a salt, prodrug or salt of a prodrug thereof.

Still another embodiment pertains to compositions for treating cancer in a mammal with measurably additive antitumorigenesis, said compositions comprising therapeutically effective amounts of valproic acid (VPA) and a peptidomimetic of the second of the three Type-1 repeats of TSP-1 or a salt, prodrug, or salt of a prodrug thereof.

Still another embodiment pertains to separate compositions to be administered together for treating cancer in a mammal with measurably additive antitumorigenesis, one comprising a therapeutically effective amount of valproic acid (VPA) and the other comprising a peptidomimetic of the second of the three Type-1 repeats of TSP-1 or a salt, prodrug, or salt of a prodrug thereof.

Still another embodiment pertains to compositions for treating cancer in a mammal with measurably additive antitumorigenesis, said compositions comprising therapeutically effective amounts of valproic acid (VPA) and N—Ac-Sar-Gly-Val-D-alloIle-Thr-Nva-Ile-Arg-Pro-NHCH₂CH₃ or a salt, prodrug, or salt of a prodrug thereof.

Still another embodiment pertains to separate compositions to be administered together for treating cancer in a mammal with measurably additive antitumorigenesis, one comprising a therapeutically effective amount of valproic acid (VPA) and the other comprising N—Ac-Sar-Gly-Val-D-alloIle-Thr-Nva-Ile-Arg-Pro-NHCH₂CH₃ or a salt, prodrug, or salt of a prodrug thereof.

Still another embodiment pertains to compositions for inhibiting tumor growth in a mammal with measurably additive antitumorigenesis, said compositions comprising therapeutically effective amounts of a HDAC inhibitor and a peptidomimetic of the second of the three Type-1 repeats of TSP-1 or a salt, prodrug, or salt of a prodrug of either or both.

Still another embodiment pertains to separate compositions to be administered together for inhibiting tumor growth in a mammal with measurably additive antitumorigenesis, one comprising a therapeutically effective amount of a HDAC inhibitor or a salt, prodrug, or salt of a prodrug thereof, and the other comprising a peptidomimetic of the second of the three Type-1 repeats of TSP-1 or a salt, prodrug or salt of a prodrug thereof.

Still another embodiment pertains to compositions for inhibiting tumor growth in a mammal with measurably additive antitumorigenesis, said compositions comprising therapeutically effective amounts of a HDAC inhibitor or a salt, prodrug, or salt of a prodrug thereof and N—Ac-Sar-Gly-Val-D-alloIle-Thr-Nva-Ile-Arg-Pro-NHCH₂CH₃, or a salt, prodrug, or salt of a prodrug of either or both.

Still another embodiment pertains to separate compositions to be administered together for inhibiting tumor growth in a mammal with measurably additive antitumorigenesis, one comprising a therapeutically effective amount of a HDAC inhibitor or a salt, prodrug or salt of a prodrug thereof, and the other comprising N—Ac-Sar-Gly-Val-D-alloIle-Thr-Nva-Ile-Arg-Pro-NHCH₂CH₃ or a salt, prodrug or salt of a prodrug thereof.

Still another embodiment pertains to compositions for inhibiting tumor growth in a mammal with measurably additive antitumorigenesis, said compositions comprising therapeutically effective amounts of VPA and a peptidomimetic of the second of the three Type-1 repeats of TSP-1, or a salt, prodrug, or salt of a prodrug of either or both.

Still another embodiment pertains to separate compositions to be administered together for inhibiting tumor growth in a mammal with measurably additive antitumorigenesis, one comprising a therapeutically effective amount of VPA and the other comprising a peptidomimetic of the second of the three Type-1 repeats of TSP-1 or a salt, prodrug, or salt of a prodrug thereof.

Still another embodiment pertains to compositions for inhibiting tumor growth in a mammal with measurably additive antitumorigenesis, said compositions comprising therapeutically effective amounts of VPA and N—Ac-Sar-Gly-Val-D-alloIle-Thr-Nva-Ile-Arg-Pro-NHCH₂CH₃ or a salt, prodrug, or salt of a prodrug thereof.

Still another embodiment pertains to separate compositions to be administered together for inhibiting tumor growth in a mammal with measurably additive antitumorigenesis, one comprising a therapeutically effective amount of VPA and the other comprising N—Ac-Sar-Gly-Val-D-alloIle-Thr-Nva-Ile-Arg-Pro-NHCH₂CH₃ or a salt, prodrug, or salt of a prodrug thereof.

Still another embodiment pertains to compositions for treating fibrosarcoma in a mammal with measurably additive antitumorigenesis, said compositions comprising therapeutically effective amounts of a HDAC inhibitor and a peptidomimetic of the second of the three Type-1 repeats of TSP-1 or a salt, prodrug, or salt of a prodrug of either or both.

Still another embodiment pertains to separate compositions to be administered together for inhibiting tumor growth in a mammal with measurably additive antitumorigenesis, one comprising a therapeutically effective amount of a HDAC inhibitor or a salt, prodrug, or salt of a prodrug thereof, and the other comprising a peptidomimetic of the second of the three Type-1 repeats TSP-1 or a salt, prodrug or salt of a prodrug thereof.

Still another embodiment pertains to compositions for treating fibrosarcoma in a mammal with measurably additive antitumorigenesis, said compositions comprising therapeutically effective amounts of a HDAC inhibitor and N—Ac-Sar-Gly-Val-D-alloIle-Thr-Nva-Ile-Arg-Pro-NHCH₂CH₃ or a salt, prodrug, or salt of a prodrug of either or both.

Still another embodiment pertains to separate compositions to be administered together for treating fibro sarcoma in a mammal with measurably additive antitumorigenesis, one comprising a therapeutically effective amount of a HDAC inhibitor or a salt, prodrug or salt of a prodrug thereof, and the other comprising N—Ac-Sar-Gly-Val-D-alloIle-Thr-Nva-Ile-Arg-Pro-NHCH₂CH₃ or a salt, prodrug or salt of a prodrug thereof.

Still another embodiment pertains to compositions for treating fibrosarcoma in a mammal with measurably additive antitumorigenesis, said compositions comprising therapeutically effective amounts of VPA and a peptidomimetic of the second of the three Type-1 repeats of TSP-1 or a salt, prodrug, or salt of a prodrug thereof.

Still another embodiment pertains to separate compositions to be administered together for treating fibro sarcoma in a mammal with measurably additive antitumorigenesis, one comprising a therapeutically effective amount of VPA and the other comprising a peptidomimetic of the second of the three Type-1 repeats of TSP-1 or a salt, prodrug, or salt of a prodrug thereof.

Still another embodiment pertains to separate compositions to be administered together for treating colon cancer in a mammal with measurably additive antitumorigenesis, one comprising a therapeutically effective amount of a HDAC INHIBITOR inhibitor or a salt, prodrug, or salt of a prodrug thereof, and the other comprising a peptidomimetic of the second of the three Type-1 repeats of TSP-1 or a salt, prodrug or salt of a prodrug thereof.

Still another embodiment pertains to compositions for treating colon cancer in a mammal with measurably additive antitumorigenesis, said compositions comprising therapeutically effective amounts of a HDAC inhibitor and N—Ac-Sar-Gly-Val-D-alloIle-Thr-Nva-Ile-Arg-Pro-NHCH₂CH₃ or a salt, prodrug, or salt of a prodrug of either or both.

Still another embodiment pertains to separate compositions to be administered together for inhibiting tumor growth in a mammal with measurably additive antitumorigenesis, one comprising a therapeutically effective amount of a HDAC inhibitor or a salt, prodrug or salt of a prodrug thereof, and the other comprising N—Ac-Sar-Gly-Val-D-alloIle-Thr-Nva-Ile-Arg-Pro-NHCH₂CH₃ or a salt, prodrug or salt of a prodrug thereof.

Still another embodiment pertains to compositions for treating colon cancer in a mammal with measurably additive antitumorigenesis, said compositions comprising therapeutically effective amounts of VPA and a peptidomimetic of the second of the three Type-1 repeats of TSP-1 or a salt, prodrug, or salt of a prodrug of either or both.

Still another embodiment pertains to separate compositions to be administered together for treating colon cancer in a mammal with measurably additive antitumorigenesis, one comprising a therapeutically effective amount of VPA and the other comprising a peptidomimetic of the second of the three Type-1 repeats of TSP-1 or a salt, prodrug, or salt of a prodrug thereof.

Still another embodiment pertains to compositions for treating colon cancer in a mammal with measurably additive antitumorigenesis, said compositions comprising therapeutically effective amounts of VPA and N—Ac-Sar-Gly-Val-D-alloIle-Thr-Nva-Ile-Arg-Pro-NHCH₂CH₃, or a salt, prodrug, or salt of a prodrug thereof.

Still another embodiment pertains to separate compositions to be administered together for treating colon cancer in a mammal with measurably additive antitumorigenesis, one comprising a therapeutically effective amount of VPA and the other comprising N—Ac-Sar-Gly-Val-D-alloIle-Thr-Nva-Ile-Arg-Pro-NHCH₂CH₃ or a salt, prodrug, or salt of a prodrug thereof.

Still another embodiment pertains to methods for treating angiogenic diseases in a mammal with measurably additive antitumorigenesis, said methods comprising administering to the mammal therapeutically effective amounts of a HDAC inhibitor and a peptidomimetic of the second of the three Type-1 repeats of TSP-1 or a salt, prodrug or salt of a prodrug of either or both.

Still another embodiment pertains to methods for treating angiogenic diseases in a mammal comprising administering to the mammal a therapeutically effective amount of a peptidomimetic of the second of the three Type-1 repeats of TSP-1 or a salt, prodrug or salt of a prodrug thereof at least once per day for at least fourteen days and a HDAC inhibitor or a salt, prodrug or salt of a prodrug thereof on at least days one, three, eight and ten.

Still another embodiment pertains to methods for treating angiogenic diseases in a mammal with measurably additive antitumorigenesis, said methods comprising administering to the mammal therapeutically effective amount of a HDAC inhibitor and N—Ac-Sar-Gly-Val-D-alloIle-Thr-Nva-Ile-Arg-Pro-NHCH₂CH₃ or a salt, prodrug or salt of a prodrug of either or both.

Still another embodiment pertains to methods for treating angiogenic diseases in a mammal comprising administering to the mammal a therapeutically effective amount of N—Ac-Sar-Gly-Val-D-alloIle-Thr-Nva-Ile-Arg-Pro-NHCH₂CH₃ or a salt, prodrug or salt of a prodrug thereof at least once per day for at least fourteen days and a HDAC inhibitor or a salt, prodrug or salt of a prodrug thereof on at least days one, three, eight and ten.

Still another embodiment pertains to methods for treating angiogenic diseases in a mammal with measurably additive antitumorigenesis, said methods comprising administering to the mammal therapeutically effective amounts of VPA and a peptidomimetic of the second of the three Type-1 repeats of TSP-1 or a salt, prodrug or salt of a prodrug thereof.

Still another embodiment pertains to methods for treating angiogenic diseases in a mammal comprising administering to the mammal a therapeutically effective amount of a peptidomimetic of the second of the three Type-1 repeats of TSP-1 or a salt, prodrug or salt of a prodrug thereof at least once per day for at least fourteen days and VPA on at least days one, three, eight and ten.

Still another embodiment pertains to methods for treating angiogenic diseases in a mammal with measurably additive antitumorigenesis, said methods comprising administering to the mammal therapeutically effective amounts of VPA and N—Ac-Sar-Gly-Val-D-alloIle-Thr-Nva-Ile-Arg-Pro-NHCH₂CH₃ or a salt, prodrug or salt of a prodrug thereof.

Still another embodiment pertains to methods for treating angiogenic diseases in a mammal comprising administering to the mammal a therapeutically effective amount of N—Ac-Sar-Gly-Val-D-alloIle-Thr-Nva-Ile-Arg-Pro-NHCH₂CH₃ or a salt, prodrug or salt of a prodrug thereof at least once per day for at least fourteen days and VPA on at least days one, three, eight and ten.

Still another embodiment pertains to methods for inhibiting tumor growth in a mammal with measurably additive antitumorigenesis, said methods comprising administering to the mammal therapeutically effective amounts of a HDAC inhibitor and a peptidomimetic of the second of the three Type-1 repeats of TSP-1 or a salt, prodrug or salt of a prodrug of either or both.

Still another embodiment pertains to methods for inhibiting tumor growth in a mammal comprising administering to the mammal a therapeutically effective amount of a peptidomimetic of the second of the three Type-1 repeats of TSP-1 or a salt, prodrug or salt of a prodrug thereof at least once per day for at least fourteen days and a HDAC inhibitor or a salt, prodrug or salt of a prodrug thereof on at least days one, three, eight and ten.

Still another embodiment pertains to methods for inhibiting tumor growth in a mammal with measurably additive antitumorigenesis, said methods comprising administering to the mammal therapeutically effective amounts of a HDAC inhibitor and N—Ac-Sar-Gly-Val-D-alloIle-Thr-Nva-Ile-Arg-Pro-NHCH₂CH₃ or a salt, prodrug or salt of a prodrug thereof.

Still another embodiment pertains to methods for inhibiting tumor growth in a mammal comprising administering to the mammal a therapeutically effective amount of N—Ac-Sar-Gly-Val-D-alloIle-Thr-Nva-Ile-Arg-Pro-NHCH₂CH₃ or a salt, prodrug or salt of a prodrug thereof at least once per day for at least fourteen days and a HDAC inhibitor or a salt, prodrug or salt of a prodrug thereof on at least days one, three, eight and ten.

Still another embodiment pertains to methods for inhibiting tumor growth in a mammal with measurably additive antitumorigenesis, said methods comprising administering to the mammal therapeutically effective amounts of VPA and a peptidomimetic of the second of the three Type-1 repeats TSP-1 or a salt, prodrug or salt of a prodrug of either or both.

Still another embodiment pertains to methods for inhibiting tumor growth in a mammal comprising administering to the mammal a therapeutically effective amount of a peptidomimetic of the second of the three Type-1 repeats of TSP-1 or a salt, prodrug or salt of a prodrug thereof at least once per day for at least fourteen days and VPA on at least days one, three, eight and ten.

Still another embodiment pertains to methods for inhibiting tumor growth in a mammal with measurably additive antitumorigenesis, said methods comprising administering to the mammal therapeutically effective amounts of VPA and N—Ac-Sar-Gly-Val-D-alloIle-Thr-Nva-Ile-Arg-Pro-NHCH₂CH₃ or a salt, prodrug or salt of a prodrug thereof.

Still another embodiment pertains to methods for inhibiting tumor growth in a mammal comprising administering to the mammal a therapeutically effective amount of N—Ac-Sar-Gly-Val-D-alloIle-Thr-Nva-Ile-Arg-Pro-NHCH₂CH₃ or a salt, prodrug or salt of a prodrug thereof at least once per day for at least fourteen days and VPA on at least days one, three, eight and ten.

Still another embodiment pertains to methods for treating fibrosarcoma in a mammal with measurably additive antitumorigenesis, said methods comprising administering to the mammal therapeutically effective amounts of a HDAC inhibitor and a peptidomimetic of the second of the three Type-1 repeats of TSP-1 or a salt, prodrug or salt of a prodrug of either or both.

Still another embodiment pertains to methods for treating fibrosarcoma in a mammal comprising administering to the mammal a therapeutically effective amount of a peptidomimetic of the second of the three Type-1 repeats of TSP-1 or a salt, prodrug or salt of a prodrug thereof at least once per day for at least fourteen days and a HDAC inhibitor or a salt, prodrug or salt of a prodrug thereof on at least days one, three, eight and ten.

Still another embodiment pertains to methods for treating fibrosarcoma in a mammal with measurably additive antitumorigenesis, said methods comprising administering to the mammal therapeutically effective amounts of a HDAC inhibitor and N—Ac-Sar-Gly-Val-D-alloIle-Thr-Nva-Ile-Arg-Pro-NHCH₂CH₃ or a salt, prodrug or salt of a prodrug of either or both.

Still another embodiment pertains to methods for treating fibrosarcoma in a mammal comprising administering to the mammal a therapeutically effective amount of N—Ac-Sar-Gly-Val-D-alloIle-Thr-Nva-Ile-Arg-Pro-NHCH₂CH₃ or a salt, prodrug or salt of a prodrug thereof at least once per day for at least fourteen days and a HDAC inhibitor or a salt, prodrug or salt of a prodrug thereof on at least days one, three, eight and ten.

Still another embodiment pertains to methods for treating fibrosarcoma in a mammal with measurably additive antitumorigenesis, said methods comprising administering to the mammal therapeutically effective amounts of VPA and a peptidomimetic of the second of the three Type-1 repeats of TSP-1 or a salt, prodrug or salt of a prodrug thereof.

Still another embodiment pertains to methods for treating fibrosarcoma in a mammal comprising administering to the mammal a therapeutically effective amount of a peptidomimetic of the second of the three Type-1 repeats of TSP-1 or a salt, prodrug or salt of a prodrug thereof at least once per day for at least fourteen days and VPA on at least days one, three, eight and ten.

Still another embodiment pertains to methods for treating fibrosarcoma in a mammal with measurably additive antitumorigenesis, said methods comprising administering to the mammal therapeutically effective amounts of VPA and N—Ac-Sar-Gly-Val-D-alloIle-Thr-Nva-Ile-Arg-Pro-NHCH₂CH₃ or a salt, prodrug or salt of a prodrug thereof.

Still another embodiment pertains to methods for treating fibrosarcoma in a mammal comprising administering to the mammal a therapeutically effective amount of N—Ac-Sar-Gly-Val-D-alloIle-Thr-Nva-Ile-Arg-Pro-NHCH₂CH₃ or a salt, prodrug or salt of a prodrug thereof at least once per day for at least fourteen days and VPA on at least days one, three, eight and ten.

Still another embodiment pertains to methods for treating colon cancer in a mammal with measurably additive antitumorigenesis, said methods comprising administering to the mammal therapeutically effective amounts of a HDAC inhibitor and a peptidomimetic of the second of the three Type-1 repeats of TSP-1 or a salt, prodrug or salt of a prodrug of either or both.

Still another embodiment pertains to methods for treating colon cancer in a mammal comprising administering to the mammal a therapeutically effective amount of a peptidomimetic of the second of the three Type-1 repeats of TSP-1 or a salt, prodrug or salt of a prodrug thereof at least once per day for at least fourteen days and a HDAC inhibitor or a salt, prodrug or salt of a prodrug thereof on at least days one, three, eight and ten.

Still another embodiment pertains to methods for treating colon cancer in a mammal with measurably additive antitumorigenesis, said methods comprising administering to the mammal therapeutically effective amounts of a HDAC inhibitor and N—Ac-Sar-Gly-Val-D-alloIle-Thr-Nva-Ile-Arg-Pro-NHCH₂CH₃ or a salt, prodrug or salt of a prodrug of either or both.

Still another embodiment pertains to methods for treating colon cancer in a mammal comprising administering to the mammal a therapeutically effective amount of N—Ac-Sar-Gly-Val-D-alloIle-Thr-Nva-Ile-Arg-Pro-NHCH₂CH₃ or a salt, prodrug or salt of a prodrug thereof at least once per day for at least fourteen days and a HDAC inhibitor or a salt, prodrug or salt of a prodrug thereof on at least days one, three, eight and ten.

Still another embodiment pertains to methods for treating colon cancer in a mammal with measurably additive antitumorigenesis, said methods comprising administering to the mammal therapeutically effective amounts of VPA and a peptidomimetic of the second of the three Type-1 repeats of TSP-1 or a salt, prodrug or salt of a prodrug thereof.

Still another embodiment pertains to methods for treating colon cancer in a mammal comprising administering to the mammal a therapeutically effective amount of a peptidomimetic of the second of the three Type-1 repeats of TSP-1 or a salt, prodrug or salt of a prodrug thereof at least once per day for at least fourteen days and VPA on at least days one, three, eight and ten.

Still another embodiment pertains to methods for treating colon cancer in a mammal with measurably additive antitumorigenesis, said methods comprising administering to the mammal therapeutically effective amounts of VPA and N—Ac-Sar-Gly-Val-D-alloIle-Thr-Nva-Ile-Arg-Pro-NHCH₂CH₃ or a salt, prodrug or salt of a prodrug thereof.

Still another embodiment pertains to methods for treating colon cancer in a mammal comprising administering to the mammal a therapeutically effective amount of N—Ac-Sar-Gly-Val-D-alloIle-Thr-Nva-Ile-Arg-Pro-NHCH₂CH₃ or a salt, prodrug or salt of a prodrug thereof at least once per day for at least fourteen days and VPA on at least days one, three, eight and ten.

### DETAILED DESCRIPTION OF THE INVENTION

Variable moieties herein are represented by identifiers (capital letters with numerical and/or alphabetical superscripts) and may be specifically embodied.

Proper valences are maintained for all moieties and combinations thereof of the compounds of this invention.

The term “treating,” as used herein, means at least sustaining and preferably reversing the course of a disease or adverse physiological event.

The term “angiogenesis,” as used herein, means formation of new blood vessels.

The term “cancer,” as used herein, means growth of tumor cells which interfere with the growth of healthy cells. Cancers include, but are not limited to, fibrosarcoma and gastrointestinal cancer such as gastric cancer, colon cancer and the like.

The term “mammal,” as used herein, means a particular class of vertebrate.

The term “measurably additive antiangiogenic effect,” as used herein means greater antitumorigenesis than obtained from use of either a HDAC inhibitor or a peptidomimetic of the second of the three Type-1 repeats of TSP-1 or a salt, prodrug or salt of a prodrug of either or both.

The term “thrombospondin-1,” as used herein, means an antiangiogenic protein which functions by inhibiting endothelial cell proliferation, thereby inducing apoptosis (programmed cell death).

The term “antitumorigenesis,” as used herein, means inhibition of tumor growth.

The term “peptidomimetic of the second of the three Type-1 repeats of TSP-1,” as used herein, means parent peptide Gly-Val-Ile-Thr-Arg-Ile-Arg, the N-terminus Gly of which is capped with R¹-Sar, the Ile of which is replaced with D-Ile or D-alloIle, the Arg of which is replaced with Nva or Gln and the terminal Arg of which is replaced with Pro-R², wherein R¹ is hydrogen or an N-terminus prodrug-forming moiety, and R² is hydrogen or a C-terminus prodrug-forming moiety. N—Ac-Sar-Gly-Val-D-alloIle-Thr-Nva-Ile-Arg-Pro-NHCH₂CH₃ may also be referred to as ABT-510.

Compounds of this invention contain amino acids having asymmetrically substituted carbon atoms in the L- or D-configuration, wherein amino acids having the L-configuration are those which occur naturally. Atoms with an excess of one configuration over the other are assigned the configuration which is present in the higher amount, preferably an excess of about 85%-90%, more preferably an excess of about 95%-99%, and still more preferably an excess greater than about 99.5%.

The term “D-alloIle,” as used herein, means D-alloisolucyl.

The term “Arg,” as used herein, means L-argininyl.

The term “Gly,” as used herein, means L-glycyl.

The term “Gln,” as used herein, means L-glutamine.

The term “Ile,” as used herein, means L-isolucyl.

The term “Nva,” as used herein, means L-norvalinyl.

The term “Pro,” as used herein, means L-prolinyl.

The term “Sar,” as used herein, means L-sarcosyl (N-methyl-L-glycyl).

The term “Thr,” as used herein, means L-threoninyl.

The term “Val,” as used herein, means L-valyl.

The term “drugs of this invention,” as used herein, means HDAC inhibitors and peptidomimetics of the second of the three Type-1 repeats of TSP-1.

The term “prodrugs of this invention,” as used herein, means HDAC inhibitors and peptidomimetics of the second of the three Type-1 repeats of TSP-1 having attached thereto at least one prodrug-forming moiety.

Drugs of this invention may exist as an acid addition salts, basic addition salts or zwitterions. Acid addition salts are those derived from the reaction of the compounds with an acid. For example, the acetate, adipate, alginate, bicarbonate, citrate, aspartate, benzoate, benzenesulfonate, bisulfate, butyrate, camphorate, camphorsufonate, digluconate, formate, fumarate, glycerophosphate, glutamate, hemisulfate, heptanoate, hexanoate, hydrochloride, hydrobromide, hydroiodide, lactobionate, lactate, maleate, mesitylenesulfonate, methanesulfonate, naphthylenesulfonate, nicotinate, oxalate, pamoate, pectinate, persulfate, phosphate, picrate, propionate, succinate, tartrate, thiocyanate, trichloroacetic, trifluoroacetic, para-toluenesulfonate, and undecanoate salts of the drugs of this invention, and prodrugs thereof, are contemplated as being embraced by this invention. Basic addition salts of the drugs of this invention are those derived from the reaction of the same with the hydroxide, carbonate or bicarbonate of cations such as lithium, sodium, potassium, calcium and magnesium.

Drugs of this invention may be administered, for example, parenterally (intramuscularly, intraperintoneally intrasternally, intravenously subcutaneously) or transdermally.

Therapeutically effective amounts of drugs of this invention depend on the recipient of treatment, the cancer being treated and severity thereof, compositions containing them, time of administration, route of administration, duration of treatment, their potency, their rate of clearance and whether or not other drugs are co-administered. The amount of a compound of a drug of this invention used to make a composition to be administered daily to a patient in a single dose or in divided doses is from about 0.05 to about 300 mg/kg (mpk) body weight. Single dose compositions contain these amounts or a combination of submultiples thereof.

Drugs of this invention may be administered with or without an excipient. Excipients include, for example, encapsulating materials or additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents, mixtures thereof and the like.

Excipients for preparation of compositions comprising drugs of this invention to be administered parenterally or transdermally include, for example, 1,3-butanediol, castor oil, corn oil, cottonseed oil, dextrose, 5% glucose in water (D5W), germ oil, groundnut oil, isotonic sodium chloride solution (0.9% sodium chloride in water), liposomes, oleic acid, olive oil, peanut oil, Ringer's solution, safflower oil, sesame oil, soybean oil, U.S.P. or, water, mixtures thereof and the like.

Drugs of this invention containing NH, C(O)OH, OH or SH moieties may have attached thereto prodrug-forming moieties which are removed by metabolic processes and release the compounds having the freed NH, C(O)OH, OH or SH in vivo. Prodrugs of this invention may have modified or improved properties such as solubility and/or hydrophobicity, absorption in the gastrointestinal tract, bioavailability, reduction of site-of-administration irritation, tissue penetration, rate of clearance and the like.

In a preferred embodiment for the practice of this invention, the N-terminus (sarcosinyl) and the C-terminus (prolyl) of the representative peptidomimetic of the second of the three Type-1 repeats of TSP-of this invention have attached thereto an acetyl (CH₃C(O) or Ac) and an ethylamino moiety, respectively.

Other N-terminus prodrug forming groups include, but are not limited to, acetoxy (CH₃CO(O)), benzoyl (C₆H₅C(O)), benzoyloxy (C₆H₅CO(O)) and the like. Other C-terminus prodrug forming groups include, but are not limited to, ethyl, diethylamino and the like.

The following biological experimental is presented to provide what is believed to be the most useful and readily understood description of procedures and conceptual aspects of this invention.

### Cell Lines

SMS-KCNR, NMB, and NBL-W-S MYCN amplified NB cell lines were used in this study. Cells were grown at 5% CO2 in RPMI 1640 (Invitrogen, Carlsbad, Calif.) supplemented with 10% heat-inactivated fetal bovine serum, L-Glutamine, and antibiotics.

### Peptide

Lyophilized peptides with the sequence: acetyl-sarcosine-glycine-valine-D-alloisoleucine-threonine-norvaline-isoleucine-arginine-proline-ethylamide were dissolved in sterile 5% dextrose and stored at 4° C.

### Neuroblastoma Xenograft Studies

Female 4-6 week old homozygous athymic nude mice (Harlan, Madison, Wis.) were inoculated s.c. into the right flank with ^{1×107}SMS-KCNR or 1.25×10⁷ NMB cells. Once tumors were palpable (70 mm³), mice were treated 1×/day for 20 days with either acetyl-sarcosine-glycine-valine-Dalloisoleucine-threonine-norvaline-isoleucine-arginine-proline-ethylamide (IP, 40 mg/kg), VPA (IP, 400 mg/kg) or acetyl-sarcosine-glycine-valine-D-alloisoleucine-threonine-norvaline-isoleucine-arginine-proline-ethylamide and VPA (in combination). In separate experiments, treatment was started after the animals developed larger tumors>380 mm³ in size and treatment was continued for 10 days. Tumor volumes were measured twice a week and calculated using the formula: tumor volume =(length X width²)/2(25). The Student's t-test was used to compare tumor size in the control and treatment groups. Mice were sacrificed after 20 or 10 days of treatment, respectively. Animals were treated according to NIH guidelines for animal care and use, and protocols were approved by the Animal Care and Use Committee at Northwestern University.

### Results

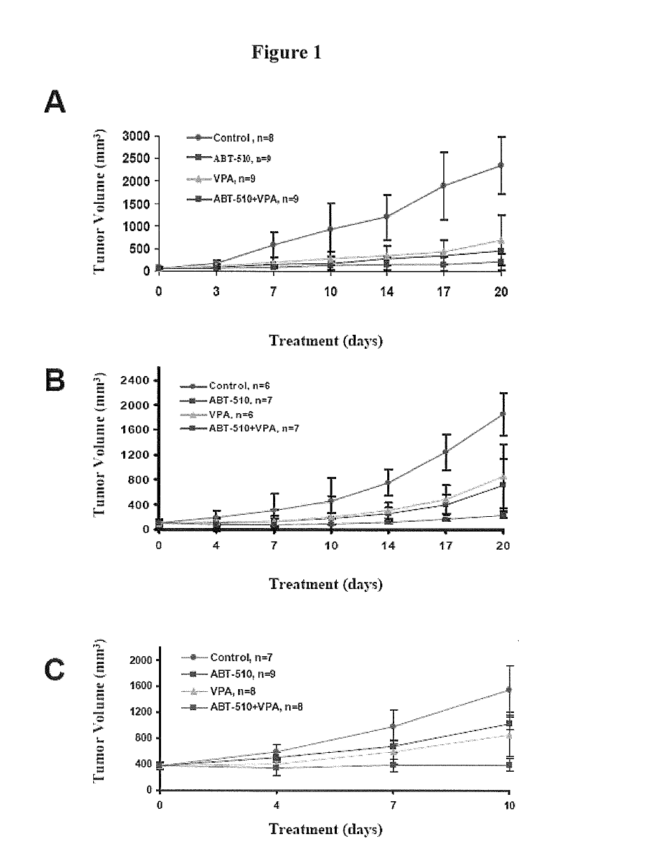

The anti-tumor effects of VPA and acetyl-sarcosine-glycine-valine-Dalloisoleucine-threonine-norvaline-isoleucine-arginine-proline-ethylamide, nude mice with xenografts (−70 mm³ in size) established from two different MYCN-amplified NB cell lines were treated with each agent alone and in combination. Tumor growth was inhibited with single agent therapy, and enhanced effects were seen with combination therapy. After 20 days of treatment with VPA combined with acetyl-sarcosine-glycine-valine-Dalloisoleucine-threonine-norvaline-isoleucine-arginine-proline-ethylamide tumor volume was reduced by 91% (p<0.001; FIG. 1A) in the SMS-KCNR model, and by 87% in the NMB xenografts (p=0.029; FIG. 1B) compared to controls.

Because the effectiveness of some anti-angiogenic strategies has been shown to be inversely related to tumor burden, we repeated our studies in a set of animals with large xenografts that were >380 mm³ in size. As shown in FIG. 1C, tumor growth was inhibited with acetyl-sarcosine-glycine-valine-Dalloisoleucine-threonine-norvaline-isoleucine-arginine-proline-ethylamide and VPA, and after 10 days of treatment tumor volumes were reduced by 33% and 44%, respectively (p<0.01 and p<0.001 respectively). Combination therapy stabilized the growth of the large NB xenografts (p<0.001).

As can be seen in the FIGS. (1A-C) both acetyl-sarcosine-glycine-valine-D-alloisoleucine-threonine-norvaline-isoleucine-arginine-proline-ethylamide and VPA inhibited NB tumor growth in vivo, and enhanced anti-tumor effects were seen with combination therapy.

The foregoing is meant to illustrate the invention and not limit it to the disclosed embodiments. Variations and changes obvious to one skilled in the art are intended to be within the scope and nature of the invention as defined in the claims.

We claim:

1. A composition for treating cancer in a mammal said composition comprising therapeutically effective amounts of a HDAC inhibitor and a peptidomimetic of the second of the three Type-1 repeats of thrombospondin-1 (TSP-1) or a salt, prodrug or salt of a prodrug of either or both.

2. A composition for treating cancer in a mammal said composition comprising therapeutically effective amounts of VPA and acetyl-sarcosine-glycine-valine-D-alloisoleucine-threonine-norvaline-isoleucine-arginine-proline-ethylamide or a salt, prodrug or salt of a prodrug of either or both.

3. The co-administration of VPA and acetyl-sarcosine-glycine-valine-D-alloisoleucine-threonine-norvaline-isoleucine-arginine-proline-ethylamide or a salt, prodrug or salt of a prodrug of either or both.

4. The co-administration of claim 3 wherein the of VPA and acetyl-sarcosine-glycine-valine-Dalloisoleucine-threonine-norvaline-isoleucine-arginine-proline-ethylamide are given in therapeutically effective amounts to treat cancer.

5. A method for treating cancer in a mammal said method comprising administering to the mammal therapeutically effective amounts of an HDAC inhibitor and a peptidomimetic of the second of the three Type-1 repeats of TSP-1 or a salt, prodrug, or salt of a prodrug thereof.

6. A method for inhibiting tumor growth in a mammal said method comprising administering to the mammal therapeutically effective amounts of VPA and acetyl-sarcosine-glycine-valine-Dalloisoleucine-threonine-norvaline-isoleucine-arginine-proline-ethylamide or a salt, prodrug or salt of a prodrug thereof.

7. The method of claim 6 wherein the tumor is neuroblastoma.
